SEQUENCE LISTING (4 sequences)

SEQ ID NO: 1 - DNA, 1298 bp, Human
ctggaggatgttgcagttcagcaaaatccaagggttggccacaggatctgctaggcaaagctgctctggatgagaaactgggaagaatgttcccattggtttgggggttgttaagccatcaggaaaaggtttggaacctgtattttaagagagcatgctatgtgattccttcctccagccctgccttcatctgtctttcacctgttcagacccttgccgtagagaatggtctcttaagagttttttactcaggaggagcaatgagcttggcttcaaggagtccactgtttcgtgaagggcattaactcagctacttggtgaactggaacctccaggcttccaaggatgacatttccctggaagaaagtagccagcagcgtcccctttgtgaaagagaggaagcctgggctccagggaagggggcaacagtccaatgagccatagcagggaatggaccgaactgtacatacaagatgaggaggggcctcgaacaacagggaagccctgtggagcacgcaggggagcctgatacgcggggaatcgcctccgtttctcaccctgacagcagggagggcttcccggagctgacagtgaggcagacatcccggatccctgttatcacaggtgcagccacgcaacttttgggttaccttcccgccacgaaatggaaatgatttcctgagggccggaaagatcatttgcatcgggcctcaggtctggggctgggaaagcaggccccggccggaaaggaagactggaggccgccgccccagaggccggaccctcgcgtcccggccgcatcccctcccggctgggtatgcgcttcgggcgcggggctccctcgcgaggctggactgggtggagcccgagagcccctccggggagctggctgccggggaggcaccggggctgcggggccgcggggcgggccgggcctggagctccgcgctagaggcggccgcagcgcaccgggcgtgggcggggccggcagcgcaagggtgaatgtcggccccgccccgacggcgctgactcagtttcaccagaaaccagggggagaaggcggccgagcccagctctccgagcaccgggtcggaagccgcgacccgagccgcgcaggaagctgggaccggaacctcggcggacccggccccacccaactcacctgcgcaggtcaccagcaccctcggaacccagaggcccgcgctctgaaggtgacccccctggggaggaaggcgatggcccctgcgaggacgatggcccgcgcccgcctcgccccggccggcatccctgccgtcgcc

SEQ ID NO: 2 - protein, 21 aa, Human
MAPARTMARARLAPAGIPAVA

SEQ ID NO: 3 - DNA, 1355 bp, Human
aaaagtgttttcaataataatgcatttatgttgattaccttgttggctatcctctactcgtgtgcctgccccactccattcccctttctagagtctgttcacgtaactcctcttccaggaagccctctttgactcccaggcttggtcaggtgtcttctctgggtttccccaggggcttccttcctctcaattctgatgctgcctgtgctaagtatttgtctccatatctgtcctgagaaccttggagccagggctcggggctgtttttgcatgggcctatcacctcctccaggaagccctccttggccctctctcaggctggatgggaaagctgtctgggctcccatcatgccctggttttctagcgacccagcctctgatccctaagcttggccttccccatcacagctttcatcactttgtgctaagcttccgctatcactactctgggccgttcctgtttggtgactcagggaccagtctggcatggccaccgctgggtccccagcattaagccctggagcatgagcccagtgaactttgaaattcaggaaggaattcacaggaaagctgcggcgaggcgggcagaaaaccacaggcttccaagaaacgcgtgtctgcctccccctcctcccaccctgaggccagtcctagagcgaaggccctcctggcacgtctgcaccaagcccaagggaagggtggcaggtgctcagcaggcagaggtgactgccccgccccgccccgccaggttctgttgggggcgaggcccgcgcaagccccgcctcttccccggcaccaggggcgggcccaggtgcgcccagggcgggggagcggccgcgcaggtgcctgccctttgcgcctgcgcccagctcgccctgcctagccaggtgcgccccgccccctgcctgcccggccaccttcgggagccgcttccaataggcgttcgccattggctctggcgacctccgcgcgttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccga

SEQ ID NO: 4 - protein, 30 aa, Human
MAQLCGLRRSRAFLALLGSLLLSGVLAADR

(19) United States
(12) Patent Application Publication
(10) Pub. No.: US 2009/0298754 A1
(43) Pub. Date: Dec. 3, 2009

(54) Hai-1 and hai-2 in cancer therapy

(76) Inventors: Wen Guo Jiang, Cardiff (GB); Christian Parr, Cardiff (GB)

Correspondence Address: KING & SCHICKLI, PLLC, 247 NORTH BROADWAY, LEXINGTON, KY, 40507, US

(21) Appl. No.: 11/659,060

(22) PCT Filed: Jul. 28, 2005
(86) PCT No.: PCT/GB05/02950

§ 371 Date: Mar. 6, 2009

(30) Foreign Application Priority Data
Aug. 4, 2004 (GB) 0417324.1

(51) Int. Cl. A61K 38/16; A61K 31/7052; C12N 5/00; C12N 1/21; C12N 1/19; C12P 21/00; C07H 21/00; C07K 16/00; A61P 35/00

(52) U.S. Cl. 514/12; 514/44 R; 435/325; 435/252.3; 435/254.2; 435/69.1; 536/24.33; 530/389.1

(57) ABSTRACT

The invention relates to a novel therapeutic composition for treating cancer, and particularly prostrate and breast cancer, the composition comprises mixture of two hepatocyte growth factor activator inhibitors HAI-1 and HAI-2.

### FIELD OF THE INVENTION

The present invention relates to a novel therapeutic composition for treating cancer, and more particularly, but not exclusively, prostate and breast cancer. The composition comprises hepatocyte growth factor activator inhibitors HAI-1 and HAI-2. Moreover, the invention relates to a method for producing said composition and its use to treat cancer.

### BACKGROUND OF THE INVENTION

Cancer is a multi-step process that includes the breakdown of the basement membrane, detachment of cancer cells from the primary tumour, invasion into the stromal layer, intravasation into blood cells, extravasation through target organ blood vessels, and the establishment and proliferation of cancer cells in remote tissues. In order for these events to take place, cancer cells must acquire the ability to migrate through and degrade extracellular matrix components. An array of growth and motility factors secreted by stromal cells have been implicated in this process, of which Hepatocyte growth factor (HGF) is one.

HGF is a pleiotropic factor initially identified as a growth factor for hepatocytes (Nakamura et al, 1987, Gohda et al, 1988 and Zarnegar et al, 1989). It is indistinguishable from scatter factor (SF), and on binding to the c-Met receptor on the surfaces of epithelial cells, HGF can disassociate epithelial colonies and scatter cells. This activity is thought to be important in the modulation of cancer cell motility and invasion, and a number of recent studies have proved that HGF/SF and Met have important roles in tumourogenesis, invasiveness of tumour cells, differentiation and tumour angiogenesis (Bellusci et al, 1994, Rong et al, 1994, Lamszus et al, 1997, Nakamura et al, 1997 and Abounader et al, 1999). Parr et al (2004) have also shown that HGF and its receptor are linked to the aggressiveness of cancers in clinical situations, such as breast cancer and prostate cancer, and high levels of HGF in tumours are associated with poor clinical outcome of patients.

HGF is secreted by mesenchymal cells as an inactive, single-chain, precursor form (scHGF) with a molecular weight of around 94 kilodaltons. To exhibit its biological function, extracellular proteolytic conversion of single-chain HGF to a two-chain heterodimeric active form is essential (Naka et al, 1992 and Gak et al, 1992). To date, five proteinases have been implicated in the activation of HGF. Amongst them, HGF activator (HGFA) exhibits the most potent activity in the processing of single-chain HGF to active HGF (Shimomura et al, 1992, Shimomura et al, 1995 and Miyazawa et al, 1993). It has been shown that HGFA is expressed at aberrantly high levels in cancers such as human breast cancer, and this expression is associated with poor clinical outcome in patients.

Given that the activation of HGF is a critical event in regulating the activity of this factor in vivo and therefore the motility of cells, it has been suggested that HGFA inhibitors could play an important role in regulating the action of HGF in cancer.

The first endogenous HGFA inhibitor (HAI) was purified from the culture-condition medium of an MKN 45 gastric carcinoma cell line (Shimomura et al, 1997). Subsequently, a second type of HAI was purified from the same cells (Kawaguchi et al 1997). These inhibitors have been designated as HAI-1 and HAI-2, respectively.

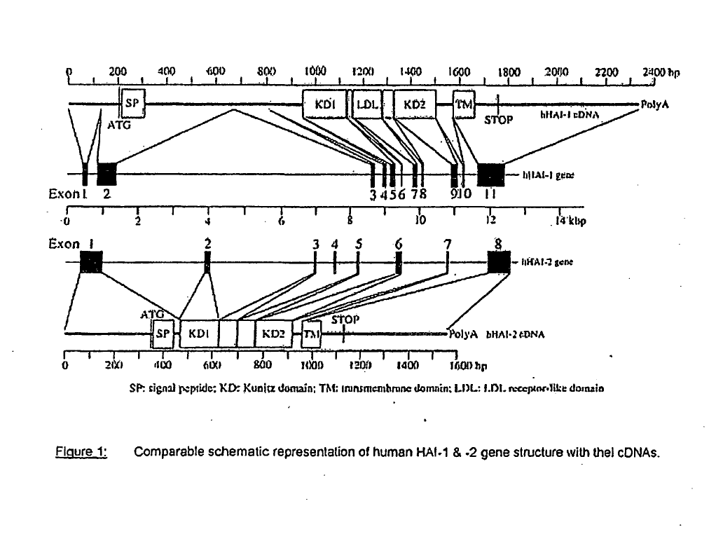

Both HAI-1 and HAI-2 have two well-defined Kunitz-type inhibitor domains (KD1 and KD2) which share a high degree of amino acid sequence identity, and the first domain appears to be responsible for the inhibition of HGFA (Shimomura et al, 1997). Additionally, each HAI has a presumed transmembrane domain (TM) near the C-terminal end, suggesting that HAI's are type I transmembrane proteins (Shimomura et al, 1997, Kawaguchi et al, 1997 and Marlor et al, 1997) (see FIG. 1) and it is thought that this structure ensures their biological activity is targeted at the cellular surface of local tissues. However, although the overall structure of the characteristic domains are similar between HAI-1 and HAI-2, the HAI-1 molecule has a low density lipoprotein (LDL) receptor-like domain that is absent in HAI-2 and HAI-2 has a testis-specific exon that is absent in HAI-1.

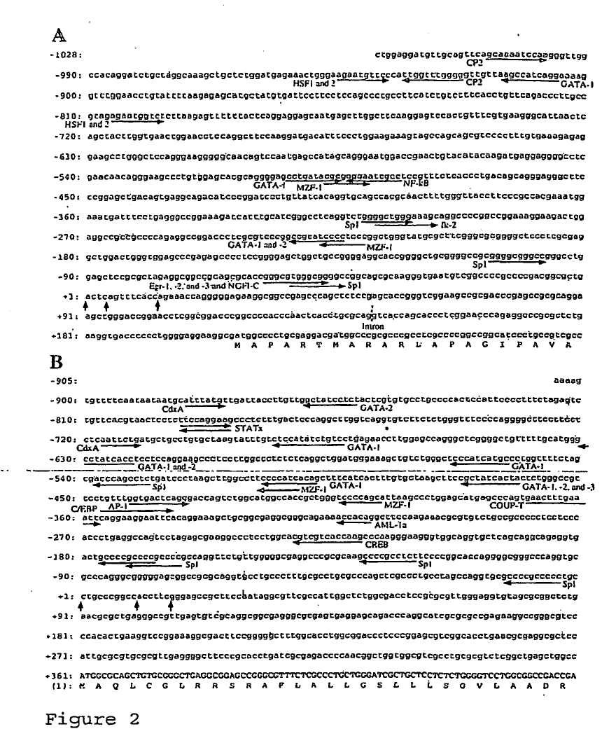

Additionally, these two proteins are mapped to different chromosomes; HAI-1 is located on chromosome 15(q 15), and HAI-2 on chromosome 19 (q 13.11). Apparently, no homologous regions between HAI-1 and HAI-2 are found in the 5′-flanking region, and potential binding sites for known transcription factors other than Sp1 and GATAs are markedly different from each other (Hoh et al, 2000) (See FIG. 2). The full genomic sequences for HAI-1 and HAI-2 have been submitted to genbank (AC 012476, AC 022086, AC 025166, and AC 022835 for HAI-1 and AC 011479 for HAI-2).

Despite this, RNA blot analyses indicate that the tissue distribution of HAI-1 is very similar to that of HAI-2, and that both genes are expressed abundantly in the placenta, kidney, pancreas and gastro-intestinal tract (Shimomura et al, 1997 and Kawaguchi et al, 1997). However, HAI-1 mRNA is only faintly detected in the testes and ovary, whereas HAI-2 is abundantly expressed in these tissues. Immunohistochemically, HAI-1 protein is localised on the lateral or baso-lateral surface of simple columnar epithelial cells covering the ducts, tubules and mucosal surfaces of various organs, including the gastro-intestinal tract (Kataoka et al, 1999). The expression of HAI-1 in colonic epithelium has also been confirmed by in situ hybridisation (Kataoka et al, 1999). In contrast, HAI-2 protein has been detected in the cytoplasm of epithelial cells and macrophage-like monocytic inflammatory cells of various tissues (Itoh et al, 2000). HAI-2 is also over-expressed in pancreatic cancer (Müller-Pillasch et al, 1998).

The difference in structural and cellular localisation of HAI-1 and HAI-2 suggest they may have distinct roles in vivo. Oberst et al (2002) have suggested that HAI-1 may suppress the growth and motility of carcinoma cells by inhibiting the generation of active HGFA. Additionally, Kawaguchi et al (1997) have suggested that HAI-1 and HAI-2 might simultaneously inhibit HGFA in vivo.

However, recently, Kataoka et al (2001) have shown that the active form of HGFA binds to HAI-1 but not HAI-2, as judged by a rigorous affinity cross-linking analysis. This specific binding of HGFA to the membrane-form of HAI-1 was further confirmed in an engineered system using China hamster ovary cells, in which only cells expressing the membrane-form of HAI-1 retained exogenously added mature HGFA. Kataoka et al concluded that HAI-1 is a cellular inhibitor of active HGFA, but the membrane form of HAI-2 is not. In support of this, studies have also shown that the cellular surface expression of HAI-1 is significantly upregulated in epithelial cells in response to tissue injury and regeneration, in which HGFA is involved, but HAI-2 expression remains unaltered (Itoh et al, 2000).

Studies into the expression of HAI-1 and HAI-2 in cancer cells have also provided conflicting results. For example, Kang et al (2003) have reported that high level expression of HAI-1 is associated with poor patient outcome in breast cancer, while Parr et al (2004) found that HAI-1 and HAI-2 were expressed at a significantly lower level in poorly differentiated breast tumours, and that overall a low level of HAI-2 in breast cancer tissues was associated with poor patient outlook. Furthermore, Kataoka et al found that expression of both HAI-1 and HAI-2 compared to corresponding normal tissues was conserved in colorectal adenocarcinomas, but lower in gastrointestinal carcinomas (2000, 1998).

Whilst structural information, expression data and cellular localisation is known for HAI-1 and HAI-2 the function of these proteins, other than their variable ability to bind HGFA, remains to be determined. Once details of their function are elucidated it will be possible to speculate on the cellular pathways that these proteins participate in and so begin to provide an explanation for why, in some instances, low levels of expression are associated with poor clinical outcome.

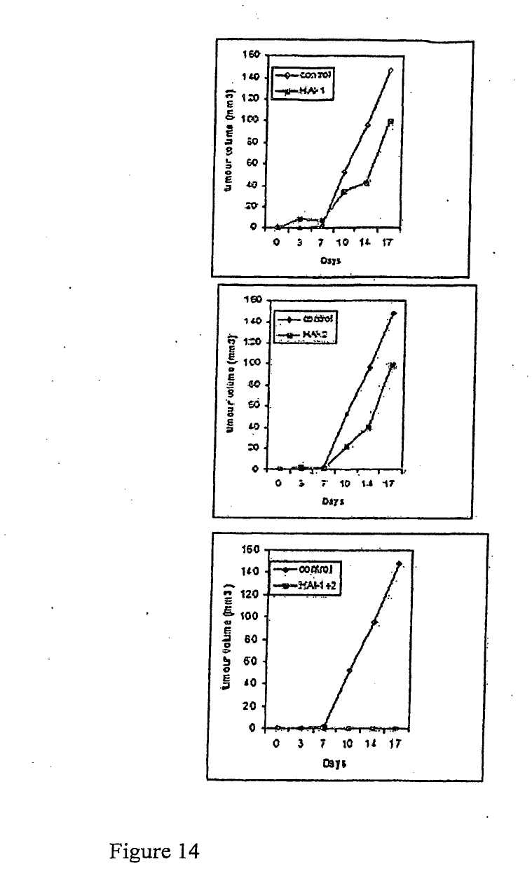

However, despite the lack of knowledge surrounding the function of these proteins we, serendipitously, chose to use them in one of our studies on cancer. Surprisingly, we found that HAI-1 and HAI-2 act synergistically to inhibit tumour growth. In particular, we have shown that peritoneal injection of either recombinant HAI-1 or HAI-2 in mice with prostrate tumour reduces tumour growth, while injection of both of these proteins simultaneously completely inhibits tumour growth (see FIG. 14).

To our knowledge, this is the first time that the synergistic effect of HAI-1 and HAI-2 has been shown, and that these proteins have been co-administered to treat cancer.

As can be seen by reference to FIG. 14, the co-administration of HAI-1 and HAI-2 resulted in no change in tumour volume, or at least, relatively no change in tumour volume since tumour volume remained at between 0 and 10 mm³ whereas untreated tumours, approximately 7 days after the study commenced, increased in growth up to 150 fold i.e. from 0 to 150 mm³. This reduced and sustained reduction in tumour volume following the co-administration of HAI-1 and HAI-2 demonstrates the remarkable synergistic effect of these two proteins to prevent or, at least, significantly reduce tumour growth. Accordingly, reference herein to the synergistic effect of these two proteins includes reference to these remarkable effects on tumour growth and thus the ability of these two proteins to prevent or significantly reduce tumour growth when compared to tumours that have not been treated with either HAI-1 or HAI-2 or when compared to tumours which have been treated with only one of HAI-1 or HAI-2.

Additionally, we believe our results are surprising because given that there is evidence that HAI-2 does not interact with HGFA and that previous studies have shown HAI-1 and/or HAI-2 are over expressed in some cancers, one would expect that administering HAI-1 and HAI-2 to a tumour model would either:

(a) have little or no effect on tumour growth compared to administering HAI-1 on its own, or
(b) increase tumour growth, respectively.

Accordingly, in one aspect of the present invention, there is provided a therapeutic composition comprising:

(a) an isolated, purified or recombinant nucleic acid molecule, encoding HAI-1 or a protein that is homologous thereto or that binds thereto under stringent hybridisation conditions; and
(b) an isolated, purified or recombinant nucleic acid molecule, encoding HAI-2 or a protein that is homologous thereto or that binds thereto under stringent hybridisation conditions.

In a preferred embodiment to the invention said homologous nucleic acid molecule is at least 80% homologous with said isolated purified or recombinant nucleic acid molecule encoding either HAI-1 or HAI-2.

In a preferred embodiment of the invention said therapeutic composition is for, or adapted for, use in treating cancer.

More particularly, the therapeutic composition is adapted for treating breast or prostrate cancer or, more preferably still, cancer of the placenta, kidney, pancreas, gastrointestinal (GI) tract, testes or ovary.

In a preferred embodiment of the invention the nucleic acid molecule may be DNA or RNA, including a cDNA or mRNA, and it may be in the form of a vector comprising a recombinant construct. Where the nucleic acid molecule is incorporated into a vector, HAI-1 and HAI-2 may be under the control of a single promoter sequence, or two different promoter sequences. Additionally, the promoter sequences may be differentially inducible. Alternatively either or both of said promoters may provide for constitutive expression of said protein(s).

In another aspect of the invention, there is provided a therapeutic composition comprising:

(a) an isolated, purified or recombinant polypeptide comprising HAI-1 protein or a functionally active fragment thereof or a polypeptide that is at least 80% homologous thereto, and
(b) an isolated, purified or recombinant polypeptide comprising HAI-2 protein or a functionally active fragment thereof or a polypeptide that is at least 80% homologous thereof.

In a preferred embodiment of the invention this therapeutic composition is for, or adapted for, use in treating cancer.

Preferably the cancer to be treated is breast cancer or prostate cancer. More preferably still, the composition is for, or adapted for, treating cancer of the pancreas, kidney, placenta, GI tract, testes or ovary.

In the aforementioned embodiments of the invention said nucleic acid molecules encoding HAI-1 or HAI-2 and said HAI-1 and HAI-2 proteins are provided in relatively equal amounts. However, the two products may be provided in unequal amounts providing that both are present in the composition.

According to a further aspect of the invention there is provided a pharmaceutical composition comprising the aforementioned therapeutic composition in combination with a suitable excipient or carrier and further the composition may be formulated for a particular application such as topical application, intravenous injection, oral administration or administration as a pessary. Formulations that are suitable for these purposes are well known to those skilled in the art.

According to a further embodiment of the invention there is provided a genetic construct comprising a nucleic acid molecule encoding HAI-1 and a nucleic acid molecule encoding HAI-2.

In a yet another further preferred embodiment of the invention said genetic construct is adapted for the expression of HAI-1 and HAI-2 in a selected system. For example, said construct may be adapted for the selective expression of HAI-1 and HAI-2 proteins in a mammalian system and therefore control sequences (such as promoters and the like) that are suitable for enabling expression of said proteins in the mammalian system are included. Such sequences are well known to those skilled in the art.

Alternatively, said genetic construct may be adapted for expression in a bacterial or yeast system and therefore the construct includes control sequences that are adapted for these purposes. Such sequences are well known to those skilled in the art.

In a preferred embodiment of the invention said genetic construct comprises at least one promoter sequence that is operationally linked to at least one of said nucleic acid molecules and, most ideally, a single promoter is linked to both said nucleic acid molecules. In an alternative embodiment, each nucleic acid molecule may be provided with its own promoter sequence. In any event, the promoter sequence may be either inductively or constitively expressed such that HAI-1 and HAI-2 proteins are controllably or constitutively produced.

More preferably still, each said nucleic acid molecule is provided with a secretion signal whereby, following expression, the relevant protein is targeted for secretion and therefore the expression product can be harvested from the cell culture medium. If this secretion-expression system is not adopted then, alternatively, expressed proteins are purified by extracting same from the relevant cell system using conventional means.

According to yet a further aspect of the invention there is provided a host cell transformed or transfected with the genetic construct of the invention.

In a preferred embodiment of the invention said host cell is of mammalian origin or bacterial origin or a yeast cell system.

In another aspect of the invention, there is provided a method for preparing a composition as described herein, which method comprises: expressing, individually or together, of HAI-1 and HAI-2 in a host cell and isolating and/or purifying the expression products.

In yet a further alternative embodiment of the invention, said therapeutic composition may be produced by isolating HAI-1 and HAI-2 from a suitable source and, in the instance where each protein is produced from a separate source, the isolated proteins are then mixed together in order to provide the therapeutic composition.

The invention also provides for a method of treating cancer by administering to an individual to be treated a composition as described herein.

In a preferred method of the invention said cancer to be treated is breast cancer or prostrate cancer and therefore said individual to be treated is a patient with either of these conditions. Alternatively, the cancer to be treated is cancer of the placenta, kidney, pancreas, GI tract, testes or ovary.

In an alternative embodiment of the invention the invention may be worked by causing the expression, or preferably the over expression, of endogenous HAI-1 and HAI-2. This may be undertaken by targeting the promoter of these genes so as to ensure that the endogenous protein is over expressed. Thus a tool for this purpose may comprise a genetic construct comprising a constitutive, or inducible, promoter that is characterised by ensuring that the gene to which it is attached is expressed either constitutively or intermittently at relatively high levels and certainly at levels high enough to ensure that a combination of HAI-1 and HAI-2 is efficient to treat cancer and, further, said construct is adapted for coupling to the nucleic acid molecule encoding HAI-1 and/or HAI-2.

According to yet a further aspect of the invention there is provided an oligonucleotide that is adapted to hybridise with at least a part of the nucleic acid molecule encoding either HAI-1 or HAI-2 and, more preferably still, there is provided a plurality of oligonucleotides for hybridising to HAI-1 or HAI-2. More preferably still, there is provided a five prime and three prime oligonucleotide for binding to HAI-1 or HAI-2 in order to achieve the effective amplification of same with a view to manufacturing a supply of HAI-1 or HAI-2 for the purpose of producing a therapeutic composition as herein described.

It will be apparent to those skilled in the art that oligonucleotides provided by this invention comprise oligonucleotides that are capable of binding to wild type or recombinant DNA encoding HAI-1 or HAI-2.

In summary our current study has shown that a combination of HAI-1 and HAI-2 is an effective inhibitor of tumour growth and thus these substances, when co-administered, have particular application in treating cancer.

According to a further aspect of the invention there is provided an antibody raised against HAI-1 or HAI-2.

Antibodies in accordance with the invention have particular use in the provision of a growth promoting factor. As a skilled man will be aware from the disclosure herein a combination of HAI-1 and HAI-2 completely inhibits tumour growth and therefore it follows that antibodies, or a combination of antibodies raised against HAI-1 or HAI-2 have utility in blocking the activity of these two agents and thus promoting cellular growth. It therefore follows that the use of antibodies to HAI-1 and HAI-2 in a suspension, or more ideally, solution has particular application in the development of a growth promoting culture medium.

According to a further aspect of the invention there is therefore provided a growth promoting factor comprising an antibody raised against HAI-1 and an antibody raised against HAI-2.

In a preferred embodiment of the invention said antibodies are monoclonal or more preferably still humanised monoclonal antibodies.

The invention will now be described by way of the following example and with reference to the following figures where:

FIG. 1 shows the organisation of HAI-1 (upper portion) and HAI-2 (lower portion) genes with corresponding cDNAs. The locations of exons are indicated by a black box with the exon number. The portions of cDNAs corresponding to each exon with approximate DNA sizes are also indicated (Taken from Itoh et al 2000);

FIG. 2 shows the nucleotide sequences of 5′ flanking region of human HAI-1 (A) and HAI-2 (B) genes. Nucleotide residues are shown in minus numbers from the transcription start site. Possible transcription start sites including minor ones are indicated in vertical arrows. The potential binding sites of known transcription factors are indicated by horizontal arrows with their names. HAI-1 has a first intron in the 5′ flanking region at the position shown by the darkened line (Taken from Itoh et al, 2000);

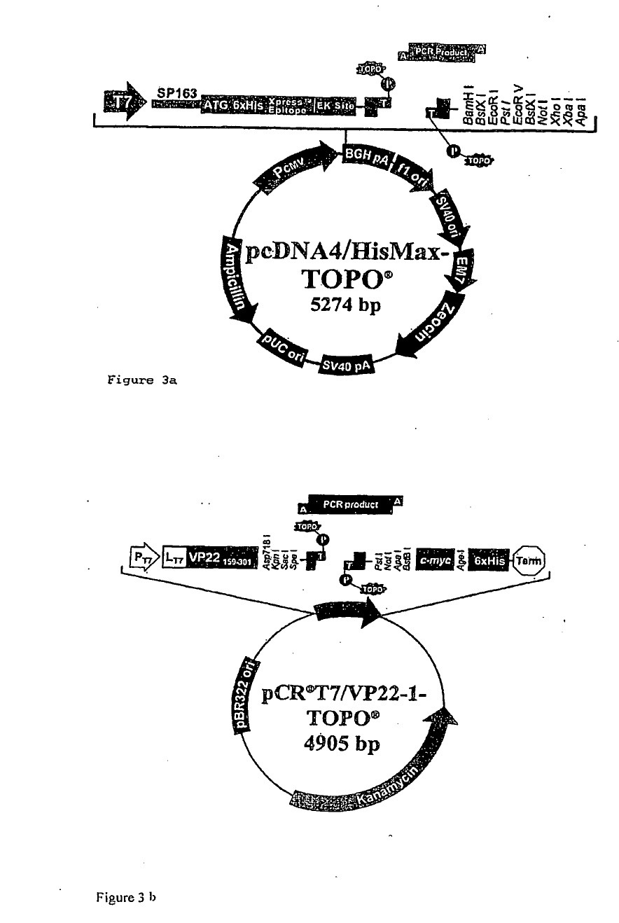

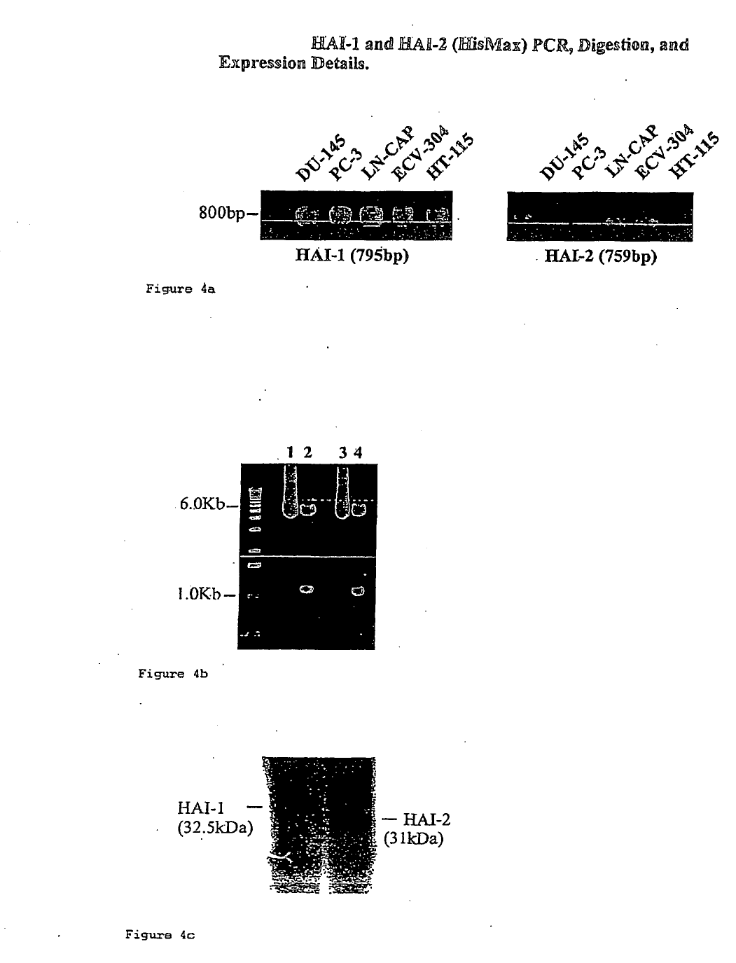

FIG. 3(a) shows a map of pcDNA 4/HisMax-TOPO. This diagram shows the features of this 5.27 Kb vector. The cloning site is located between bases 1184-1185;

FIG. 3(b) shows a map of pCR-T7/VP-22-1-TOPO. This diagram summarises the features of the VP-22 vector. The vector is 4.9 Kb in size, and the cloning site is located between bases 597-598;

FIG. 4(a) shows HAI-1 and HAI-2 digestion and expression details. The PCT products representing the amplified HAI-1 and HAI-2 target sequences, were strongest/cleanest in the LN-CAP and ECV-304 samples, respectively. These PCR products were used directly for TA cloning;

FIG. 4(b) The purified HisMax plasmids, containing the appropriate HAI inserts (1 & 3), were digested with Hind III and Eco RV restriction enzymes to release the cloned HAI sequence (2 & 4). (Note: Hind III to HAI sequence=275 bp, and Eco RV to HAI=45 bp, thus, extra 0.32 Kb):

### 1. HisMax (5.27 Kb)+HAI-1 (0.8 Kb)=6.07 Kb

### 3. HisMax (5.27 Kb)+HAI-2 (0.76 Kb)=6.03 Kb

2. HAI-1 (0.8 Kb)+extras 0.32 Kb=1.12 Kb
4. HAI-2 (0.76 Kb)+extra 0.32 Kb=1.08 Kb);

FIG. 4(c) Cell-free expression of HAI-1 and HAI-2. The HisMax vector, containing HAI-1, produces a protein of 300 amino acids (900 bp), with a molecular weight of 32.5 kDa. A 286 amino acid (860 bp) protein is produced with HAI-2, at 31 kDa;

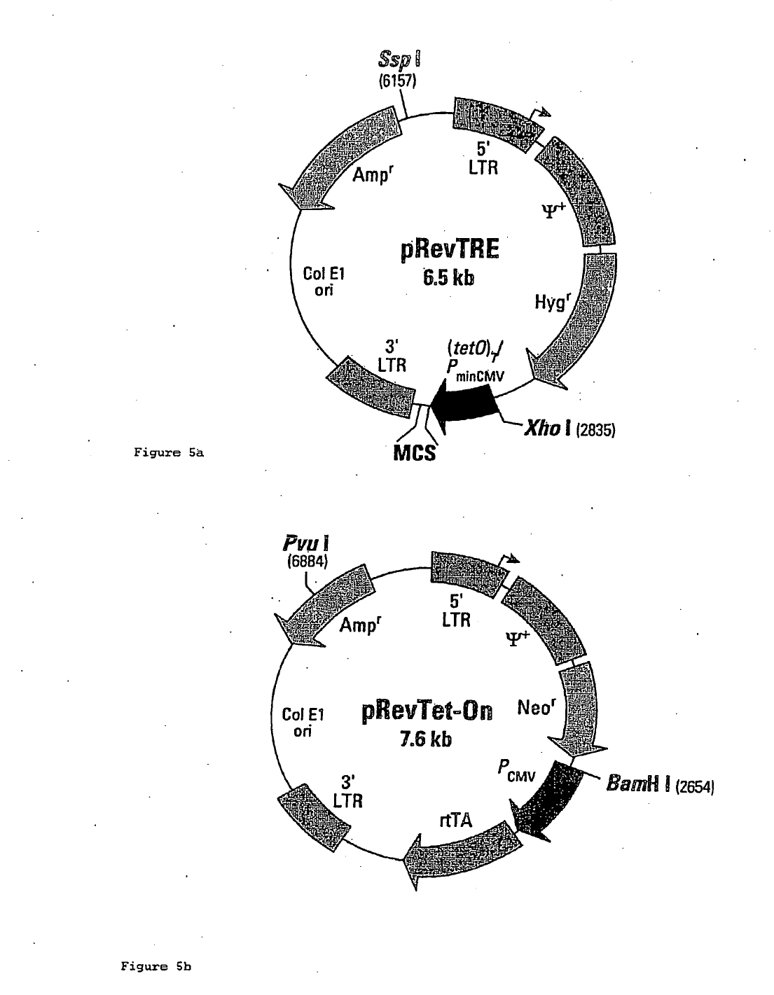

FIG. 5(a) shows a map of the pRevTRE vector;

FIG. 5(b) shows a map of the pRevTet-On vector;

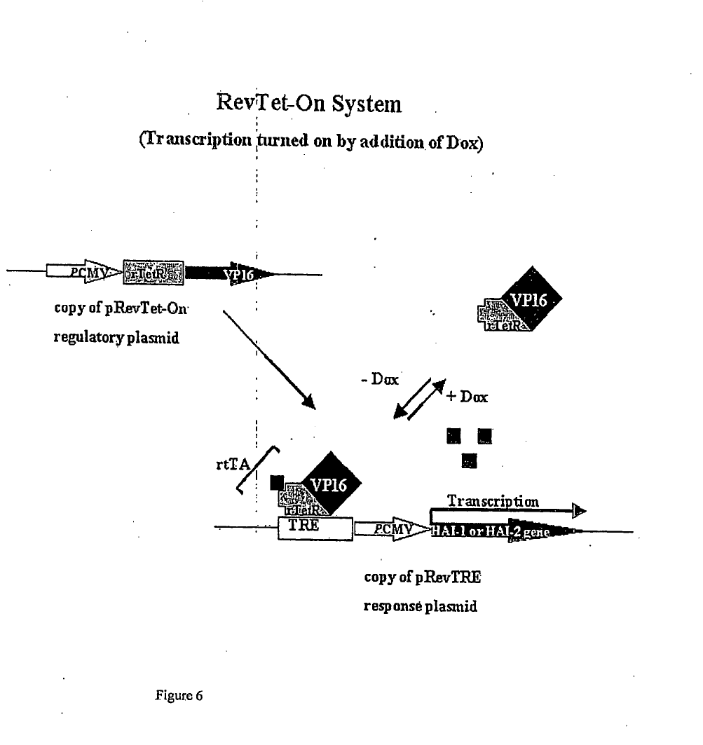

FIG. 6 shows the mechanism of RevTet-On gene expression. The tet response element (TRE) is located upstream of the minimal immediate early promoter of cytomegalovirus (PCMV), which is silent in the absence of activation. The reverse tetracycline-controlled transactivator (rtTA) binds the TRE, thereby activating HAI transcription, in the presence of Doxycycline (Dox);

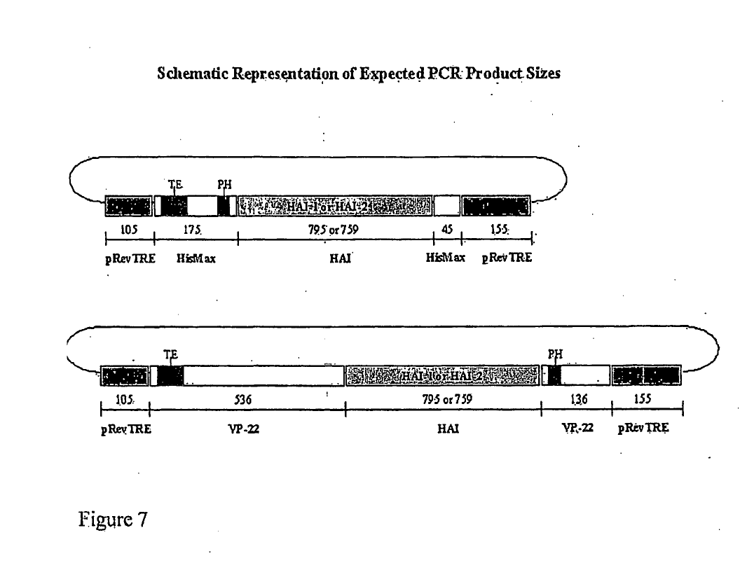

FIG. 7 shows the amplification of target sequences with either the pRevTRE, VP-22, HisMax or HAI primers (as shown in Table 9.1), generates PCR products of variable sizes. For example, use of the VP-22 forward and reverse primers for HAI-1, results in products of 1467 bp (536+795+136). (Note: TE is the specific translation enhancer, while, PH represents the position of the polyhistidine tag.);

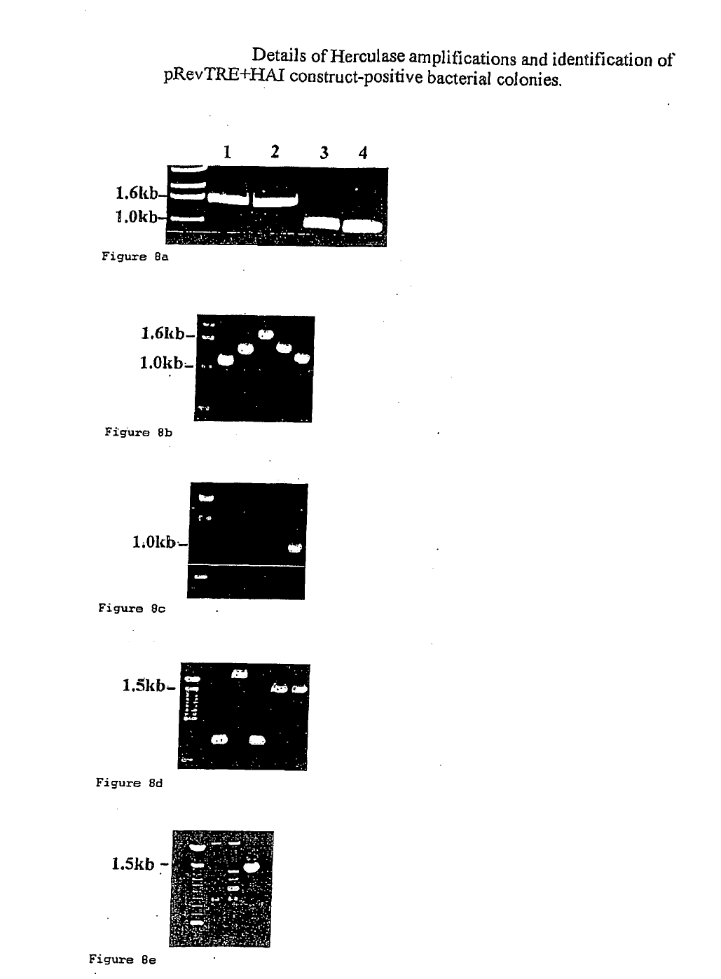

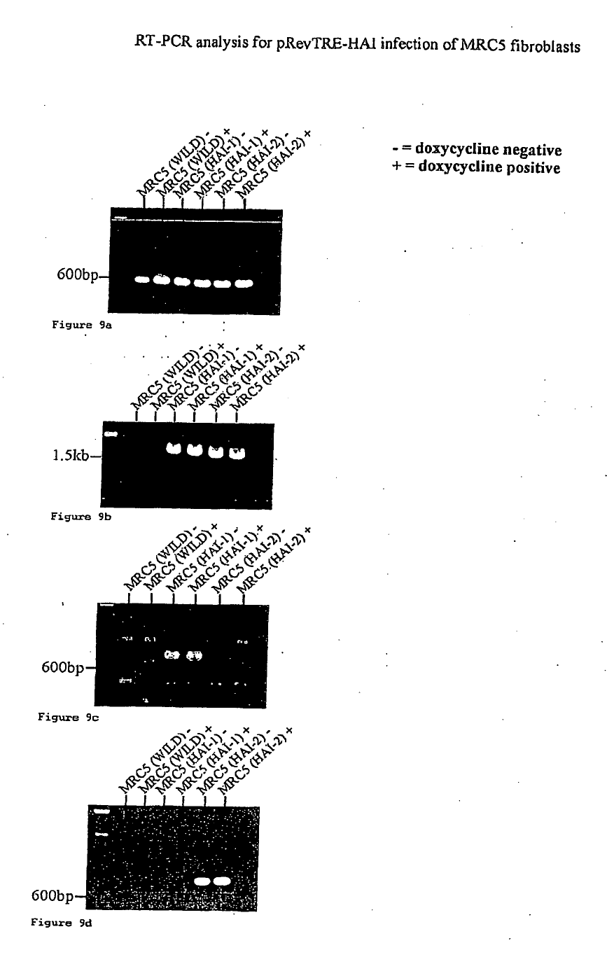

FIG. 8(a) shows Herculase system amplifications, using the HisMax and VP-22 sets of primers (see Table 9.1). Bands produced were of the expected size (see FIG. 9.3 for explanation of sizes):

### 1. VP-22+HAI-1 (672+795)=1.47 Kb

### 2. VP-22+HAI-2 (672+759)=1.43 Kb)

### 3. HisMax+HAI-1 (220+795)=1.02 Kb

### 4. HisMax+HAI-2 (220+759)=0.98 Kb;

FIG. 8(b) shows pRevTRE+HAI-1 (HisMax) bacterial colony check, with pRevTRE set of primers. 5 bands produced. Expected size (260 bp+1.02 Kb)≈1.28 Kb;

FIG. 8(c) shows confirmation of correct orientation of HAI-1 sequence within the pRevTRE vector. Users pRevTRE forward primer and HAI-1 revers primer.

Only 1 of the 5 above colonies, contained HAI-1 correctly integrated into pRevTRE. Expected size (105+175+795)=1.08 Kb;

FIG. 8(d) show pRevTRE+HAI-2 (VP.22) bacterial colony check, with pRevTRE primer set. 3 colonies produced bands within the correct range. Expected size (1.43 Kb+260 bp)=1.69 Kb;

FIG. 8(e) shows confirmation of correct orientation of HAI-2 within pRevTRE vector. Examined with pRevTRE forward primer and HAI-2 reverse primer. Only 1 of the 3 colonies above contained HAI-2 integrated into the pRevTRE vector in the correct position. Expected size (105+536+759)=1.4 Kb;

FIG. 9(a) shows β-actin quality control check;

FIG. 9(b) shows pRevTRE primer set used to confirm transduction of MRC5 fibroblasts with pRevTRE+HAI constructs;

FIG. 9(c) HAI-1 primer set shows that HAI-1 only present in MRC5 cells transduced with pRevTRE+HAI-1;

FIG. 9(d) HAI-2 primer set shows that HAI-2 is present at a high level in the HAI-2 transduced cells. The wild type and HAI-1 transduced fibroblasts reveal a very slight band for HAI-2;

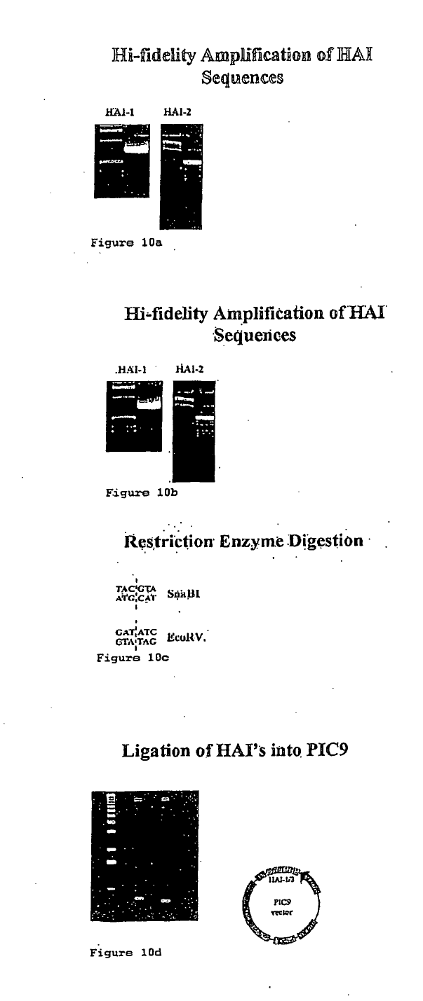

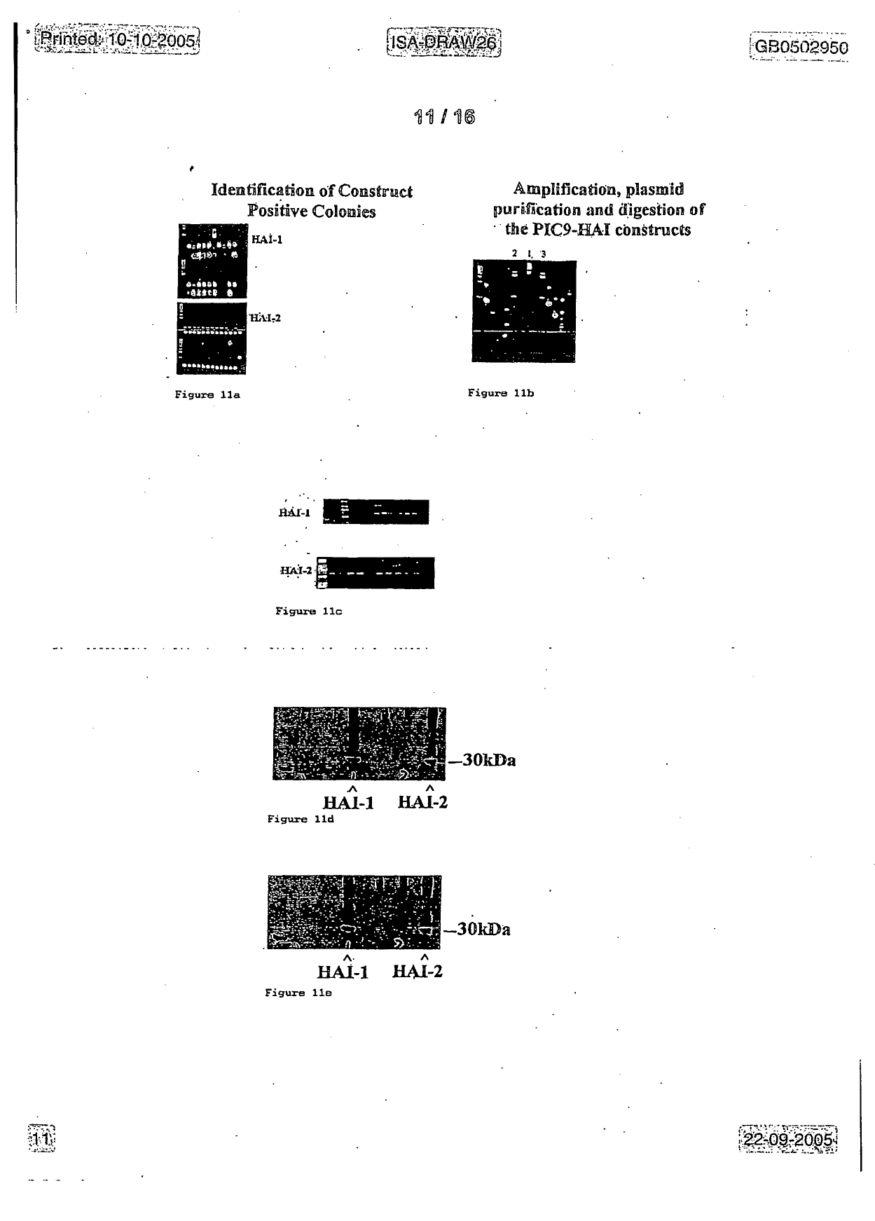

FIG. 10(a) shows a hi-fidelity PCR reaction to amplify the target sequences, from previously sequenced DNA. This ensures error free amplification of the DNA strands, due to the presence of a proof reading enzyme;

FIG. 10(b) shows a hi-fidelity PCR reaction to amplify the target sequences, from previously sequenced DNA. This ensures error free amplification of the DNA strands, due to the presence of a proof reading enzyme;

FIG. 10(c) shows the Pic9 vector was opened with the Sna B1 enzyme. The HAI DNA strands to be inserted had both ends of the strands trimmed with SnaB1 and EcoRV enzymes. These enzymes recognise and cleave specific sites on the DNA strands, resulting in ligatable blunt ends.

FIG. 10(d) Ligation of HAI's into PIC9. This ligation method requires the presence of an enzyme known as T4 DNA ligase, which catalyses the joining of two strands of DNA between the 5′-phosphate and 3′-hydroxyl groups of adjacent nucleotides;

FIG. 11(a) shows identification of construct positive colonies. PCR was used on 20-30 bacterial colonies to identify the ones that contained the PIC9 with the HAI sequence inserted. Using AOX set of PIC9 plasmid primers: No insert=500 bp and with insert=1300 bp;

FIG. 11(b) shows amplification, plasmid purification and digestion of PIC9-HAI constructs. (1) purified plasmid, (2) HAI-1 digested with Pme1 and Not1 and (3) HAI-2 digested with Pme1 and Not1. All the rest are insert orientation checks;

FIG. 11(c) shows transformation, followed by selection and subsequent identification of suitable colonies for amplification. A selection of positive yeast clones;

FIG. 11(d) Recombinant HAI-1 and HAI-2 proteins were collected from the yeast culture media and detected using antibodies developed in the Laboratory which was followed by amplification, induction of HAI-1 protein secretion and western blot analysis of HAI-1 and HAI-2 protein induction;

FIG. 11(e) shows HAI-1 and HAI-2 protein synthesis detected with antibodies developed in the Laboratory;

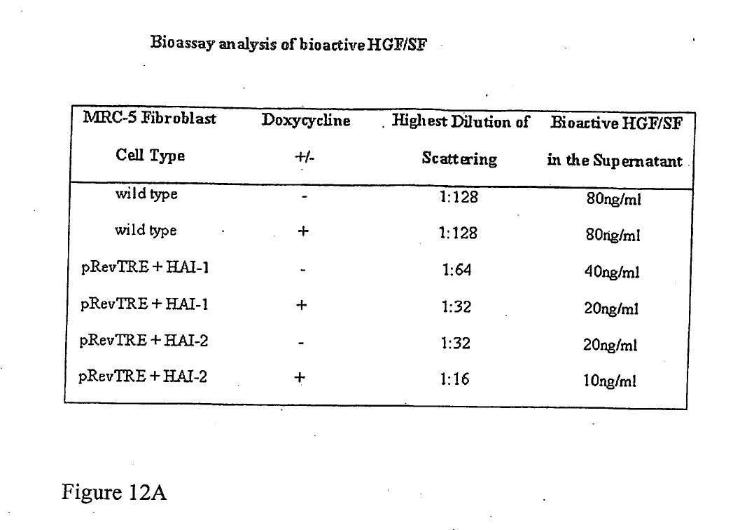

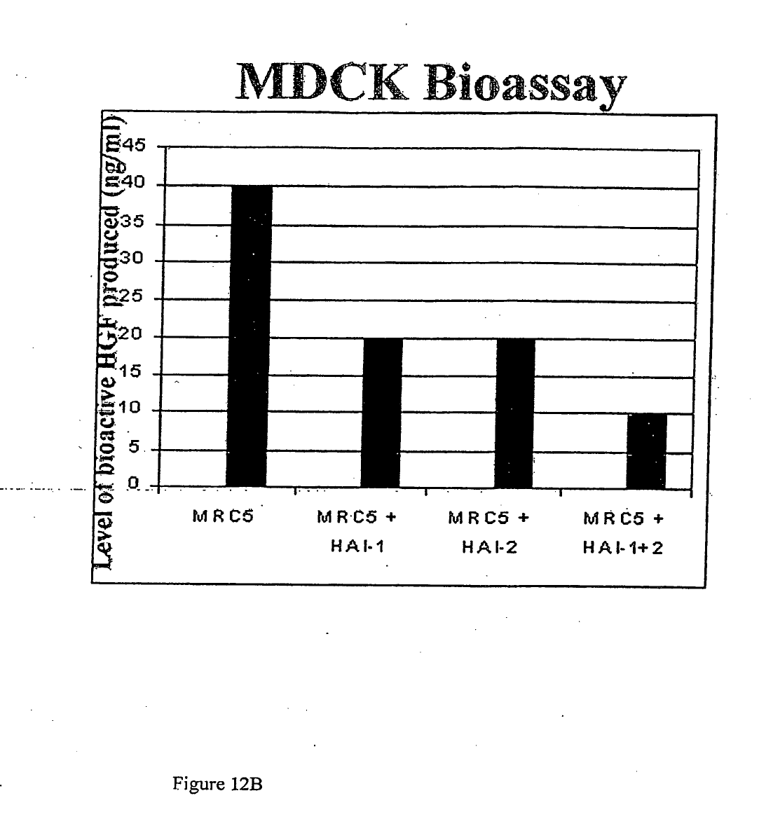

FIG. 12(a) is a bioassay analysis of bioactive HGF/SF;

FIG. 12(b) shows MDCK bioassay;

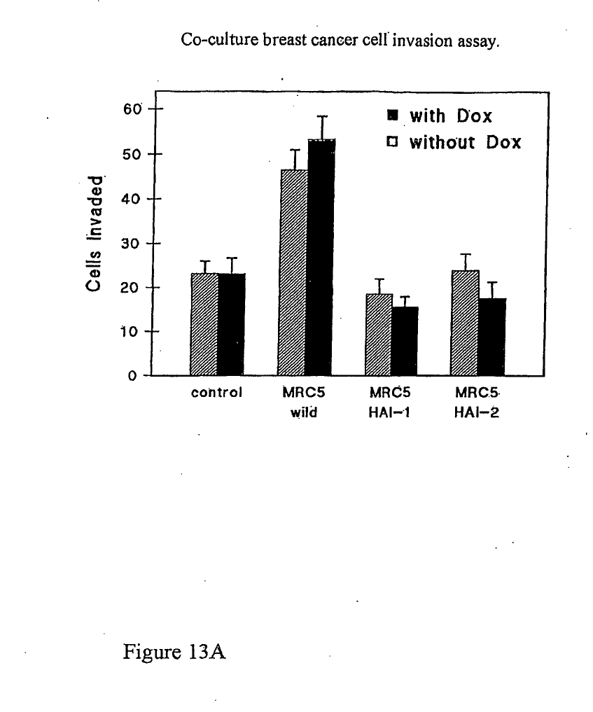

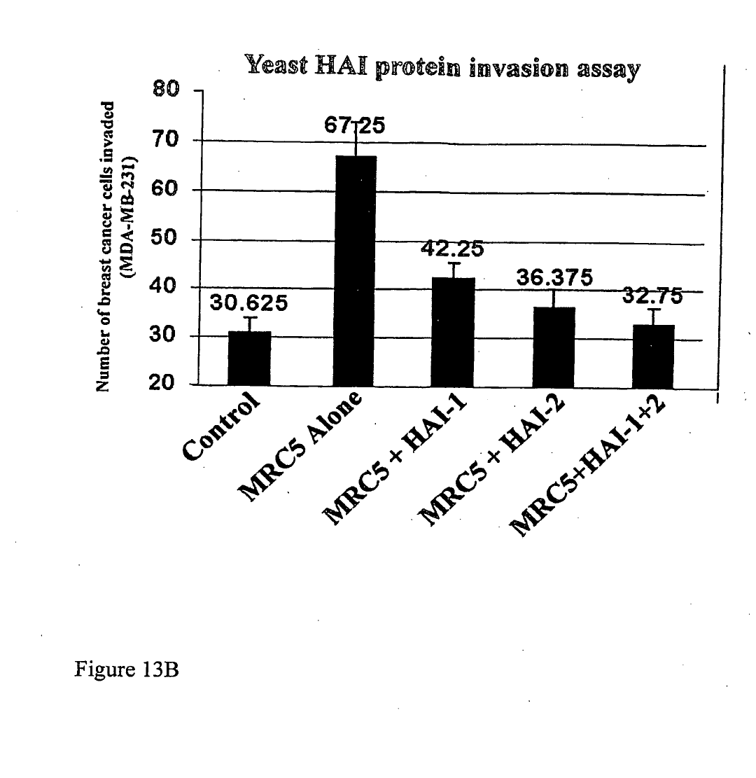

FIG. 13(a) shows a co-culture breast cancer cell invasion assay. Wild-type and transduced MRC5 fibroblasts were cultured in a 24 well plate, and utilised as a source of HGF/SF to enhance invasion of MDA-MB-231 breast cancer cells. Wild type fibroblasts significantly increased the degree of invasion compared to the control. However, the level of active HGF/SF produced by the transduced fibroblasts was significantly lower, as shown by the decrease in the number of invaded cells;

FIG. 13(b) shows a yeast HAI protein invasion assay; and

FIG. 14 shows the effect of exposure of a tumour to HAI-1, HAI-2 or both (▪) over a 17-day period.

### Amplification of Human HAIs

Human HAI-1 and HAI-2 cDNA was amplified from RNA isolated from normal human skin and human mammary tissues, using reverse transcription PCT. The correct sequence of these products were confirmed using direct DNA sequencing.

### Construction of HAIs Expression Cassettes

HAIs thus isolated were cloned into a mammalian expression vector, pCR3/His-Max (see FIG. 3), which was then used to transform mammalian cells (pcDNA4/HisMax TOPO) or chemically competent E. Coli (pCRT7/VP22-1-TOPO), respectively. The presence of the DNA inserts and the direction in bacterial colonies were verified using direction specific PCR.

Expression cassettes from the above were digested using DNA restriction enzymes (see FIG. 4). The DNA expression fragments were extracted and purified from agarose gel using a DNA extraction kit. The fragments were inserted into a retroviral vector (see FIG. 5, 6, 7), pRev-LXSN and pRev-TRE, which were similarly digested and purified using the matched registration enzymes, using T4 DNA ligase (see FIG. 8, 9). Ligated products were used to transform the JM109 strain of E. Coli, which was made chemically competent inhouse. The presence and direction of the inserts were similarly verified using direction specific PCR.

### Preparation of Bacterial Plasmid and Transfection of Cancer Cells

Following amplification of plasmid in E. Coli, plasmid DNA was extracted and purified using a plasmid preparation kit. Purified plasmid encoding HAI-1 and HAI-2 were electroporated into CHO or breast cancer cells and cells stably transfected were selected using G418 antibiotics.

### Preparation of Retroviral HAI Stock

pLXSN-HAI-1 or HAI-2, was first amplified in E. Coli. Following extraction and purification, the retroviral plasmid was electroporated into a packaging cell line, PT67. A stably transfected PT67 that produced retroviral HAI-1 or HAI-2 was obtained following selection with G418. These cells were then used to generated retroviral stocks, which was subsequently used to infect stromal fibroblasts, in which human fibroblasts cell line, MRC5, were consecutively infected for 3 days with exchange of fresh viral stock daily.

### Development of a Yeast Expression System for Both HAI-1 and HAI-2

The HAI-1/HAI-2 expression cassettes were suitably modified and re-inserted into a yeast expression vector (see FIG. 10, 11) using the T4 DNA ligase. Ligated plasmids were used to transform E. Coli, as already given above. Correct colonies were similarly selected and amplified. Purified plasmid was electroporated into a strain of yeast, using electroporator. The successful colonies were similarly selected and verified.

Yeast was grown in a yeast medium, supplemented with methanol under a special condition. The optimal condition for the strain was developed and used for the subsequent amplification to larger volume.

Yeast growth medium was centrifuged at 4° C. Following removal of the yeast, conditioned medium with recombinant HAI-1 or HAI-2 was concentrated using an ultra-filtration system which allows to retain and concentrate specified size of protein.

### HGF Bioassay

We have used an HGF bioassay (see FIGS. 12A and 12b), known as MDCK scattering assay, which is, in our view, the best way to test the bioactivity of HAI-1 and HAI-2. MDCK cells, which produced small clusters, were treated with either recombinant HAI-1, HAI-2 or their combination, together with MRC5 cells. pLXSN/HAI-1 or HAI-2 transfected MRC5 cells were also co-cultured with MDCK cells. After 24 hours, the scattering of MDCK cells were assessed, with the aid of staining using crystal violet.

### Cell Migration Assay and Cell Invasion Assay

Two methods were used to assess the effects of fibroblasts on cancer cells, cell migration assay and in vitro assays, using a co-culture system. For the migration assay, cancer cells were plated into the bottom of a chamber and allowed to reach confluence. The confluent cells were then wounded using a needle. In the upper chamber of the system, retroviral manipulated MRC5 cells (pLXSN-HAI-1 or HAI-2) were added. The migration of cancer cell wound was then monitored using a tiome-lapse video, speed of which was calculated using motion analysis software in Optimas. Cancer cells were also treated with recombinant HAI-1, HAI-2, their combination, supernatant from retroviral manipulated MRC5 cells. Cell migration was similarly determined (see FIGS. 13A and 13B).

In the in vitro invasion assay, cancer cells were added to an upper champer which was pre-coated with Matrigel, to the bottom chamber was added either recombinant HAI-1, HAI-2, their combination, supernatant from retroviral manipulated MRC5 cells, or MRC5 cells themselves. After 3 days, the invasiveness of the cells was assessed.

Detection of HAI Protein Using Western Blotting and Development of anti-HAI-1 and HAI-2 Antibodies

We first synthesised short peptides specific to human HAI-1 and HAI-2 and conjugated to KLH. The conjugate was subsequently injected into rabbits using a standard procedure to raise polyclonal antibodies. Purified antibodies were found to be specific to human HAI-1 and HAI-2, respectively. These antibodies were used to test the presence and quantity of recombinant HAI-1 and HAI-2 from the above systems.

The effects of Recombinant HAI-1 and HAI-2 in Tumour Models

Breast cancer cells MDA MD 231 or prostate cancer cells PC-3 were subcutaneously injected into the athymic nude mice (4-6 weeks old, female), using a special mixture to aid the growth of tumours. After one week, mice began to receive concentrated recombinant HAI-1, HAI-2 or their combination, daily, via the intraperitoneal route. The size of tumours and weight of mice were measured twice weekly (see FIG. 14).

### Results

As can be seen in FIGS. 12 and 13, cancer MRC5 cells had reduced motility and invasiveness when treated either with HAI-1 or HAI-2, but this was further reduced when HAI-1 and HAI-2 were administered together.

Finally, FIG. 14 shows the results of a peritoneal injection of recombinant HAI-1 and/or HAI-2 in a mouse tumour model. The results for HAI-1 and HAI-2 alone show a small reduction in tumour growth, while co-administration of these proteins results in an absolute inhibition of tumour growth.

### REFERENCES

Abounader R, Ranganathan S, Lal B, Fielding K, Book A, Dietz H, Burger P, Laterra J. Reversion of human glioblastoma malignancy by U1 small number RNA ribozyme targeting of scatter factor/hepatocyte growth factor and c-met expression. J. Natl. Cancer Inst. 91: 1548-1556, 1999 [Abstract/Free Full Text]
Bellusci S, Moens G, Gaudino G, Comoglio P, Nakamura T, Thiery J-P, Jouanneau J. Creation of a hepatocyte growth factor/scatter factor autocrine loop in carcinoma cells induces invasive properties associated with increased tumorigenicity. Oncogene, 9: 1091-1099, 1994 [Medline]
Gak E, Taylor W G, Chan A M L, Rubin J S. Processing of hepatocyte growth factor to the heterodimeric form is required for biological activity. FEBS Left., 311: 17-21 1992 [Medline]
Gohda E, Tsubouchi H, Nakayama H, Hirono S, Sakiyama O, Takahashi K, Miyazaki H, Hashimoto S, Daikuhara Y. Purification and partial characterization of hepatocyte growth factor from plasma of a patient with fulminant hepatic failure. J. Clin. Investig., 81: 414-419, 1988 [Medline]
Itoh, H., Kataoka, H., Tomita, M., Hamasuna, R., Nawa, Y., Kitamura, N. & Koono, M: (2000) Up-regulation of hepatocyte growth factor activator inhibitor type-1 but not type-2 along with regeneration of intestinal mucosa. Am. J. Physiol. 278k, 6635-6649
Itoh H, Yamauchi M, Kataoka, H, et al.: Genomic structure and chromosomal localization of the human hepatocyte growth factor activator inhibitor type 1 and 2 genes. Eur. J. Biochem. 267:3351-3359, 2000
Kang, Jung Y; Dolled-Filhart, Marisa; Ocal, Idris Tolgay; Singh, Baljit; Lin, Chen-Yong; Dickson, Robert B; Rimm, David L and Camp, Robert L. Tissue Microarray Analysis of Hepatocyte Growth Factor/Met Pathway Components Reveals A Role for Met, Matriptase, and Hepatocyte Growth Factor Activator Inhibitor 1 in the Progression of none-negative breast cancer. Cancer Research 63, 1101-1105, Mar. 1, 2003
Kataoka H, Itoh H, Uchino H, Hamasuna R, Kitamura N, Nabeshima K and Koono M. Concerved expression of hepatocyte growth factor activator inhibitor type-2/placental bikunin in human colorectal carcinomas. Cancer Lett. 148: 127-134, 2000 [ISI][Medline]
Kataoka H, Uchino H, Denda K, Kitamura N, Itoh H, Tsubouchi H, Nabeshima K and Koono M. Evaluation of hepatocyte growth factor activator inhibitor expression in normal and malignant colonic mucosa. Cancer Lett 128: 219-227, 1998 [ISI][Medline]
Kataoka, Hiroaki; Itoh, Hiroshi; Shimomura, Takeshi; Nuki, Yoshitsugu; Naganuma, Seiji and Miyazawa, Keiji. Int Arch Biosci 2001: 1036-1042
Kataoka, H., Suganuma, T., Shimomura, T, Itoh, H., Denda, K., Kitamura, N., Nabeshima, K. & Koono, M. (1999) Distribution of hepatocyte growth factor activator inhibitor type 1 (HAI-1) in human tissues: cellular surface localization of HAI-1 in simple columnar epithelium and its modulated expression in injured and regenerative tissues. J Histochem. Cytochem. 47, 673-682. [Abstract/Free Full Text)
Kawaguchi T, Qin L, Shimomura T, et al: Purification and cloning of hepatocyte growth factor activator inhibitor type 2, a Kunitz-type serine protease inhibitor. J. Biol. Chem. 272, 27558-27564. 1997
Lamszus K, Jin L, Fuchs A, Shi E, Chowdhury S, Yao Y, Polverini P J, Laterra J, Goldberg I D, Rosen E M. Scatter factor stimulates tumor growth and tumor angiogenesis in human breast cancers in the mammary fat pad of nude mice. Lab. Investig. 76: 339-353, 1997
Marlor C W, Delaria K A, Davis, G, et al: Identification and cloning of human placental bikunin, a novel serine protease inhibitor containing two Kunitz domains. J Biol. Chem. 272: 12202-12208, 1997
Miyazawa K, Shimomura T, Kitamura A, et al: Molecular cloning and sequence analysis of the cDNA for a human serine protease responsible for activation of hepatocyte growth factor. J Biol. Chem. 268: 10024-10028, 1993
Müller-Pillasch, F., Wallrapp, C., Bartels, K., Varga, G., Friess, H., Büchler, M., Adler, G. & Gresss, T. M. (1998) Cloning of a new Kunitz-type protease inhibitor with a putative transmembrane domain overexpressed in pancreatic cancer. Biochim. Biophys, Acta 1395, 88-95. [Medline]
Naka D, Ishii T, Yoshiyama Y, Miyazawa K, Hara H, Hishida T, Kitamura N. Activation of hepatocyte growth factor by proteolytic conversion of single chain form to a heterodimer. J Biol. Chem., 267: 20114-20119, 1992 [Abstract/Free Full Text]
Nakamura T, Nawa K, Ichihara A, Kaise N, Nishino T. Purification and subunit structure of hepatocyte growth factor from rat platelets. FEBS Lett., 224: 311-316, 1987 [Medline]
Nakamura T, Matsumoto K, Kirioshi A, Tan Y, Nakamura T. Induction of hepatocyte growth factor in fibroblasts by tumor-derived factors affects invasive growth of tumor cells: in vitro analysis of tumor-stomal interactions. Cancer Res, 57: 3305-3313, 1997 [Abstract]
Oberst, Michael D., Johnson, Michael D., Dickson, Robert B., Lin, Chen-Yong; Singh, Baljit; Stewart, Moria; Williams, Alastair; al-Nafussi, Awatif; Smyth, John F; Gabra, Hani and Sellar, Grant C (April 2002) Clinical Cancer Research Vol. 8, 1101-1107
Parr, Christian; Watkins, Gareth; Mansel, Robert E and Jiang, Wen G. The hepatocyte growth factor regulatory factors in human breast cancer. Clinical Cancer Research Vol 10, 202-211, January 2004
Rong S, Segal S, Anver M, Resau J H, Vande Woude G F. Invasiveness and metastasis of N1H3T3 cells induced by Met-hepatocyte growth factor/scatter factor autocrine stimulation. Proc. Natl. Acad. Sci. USA, 91: 4731-4735, 1994 [Abstract]
Shimomura T, Ochiai M, Kondo J, et al: A novel protease obtained from FBS-containing culture supernatant, that processes single-chain form hepatocyte growth factor to two chain form in serum-free culture. Cytotechnology 8:219-229, 1992
Shimomura T., Miyazawa K, Komiyama Y, et al: Activation of hepatocyte growth factor by two homologous proteases, blood-coagulation factor XIIa and hepatocyte growth factor activator. Eur. J. Biochem. 229:257-261, 1995
Shimomura T, Denda K, Kitamura A, et al: Hepatocyte growth factor activator inhibitor, a novel Kunitz-type serine protease inhibitor. J Biol. Chem. 272; 6370-6376, 1997
Zarnegar R, Michalopoulos G. Purification and biological characterization of human hepatopoietin A, a polypeptide growth factor for hepatocytes. Cancer Res., 49: 3314-3320, 1989 [Abstract]

1. A therapeutic composition comprising;
(a) An isolated, purified or recombinant polypeptide/protein comprising. HAI-1, or a functionally active fragment thereof, or a polypeptide/protein that is at least 80% homologous thereto; and
(b) An isolated, purified or recombinant polypeptide/protein comprising HAI-2, or a functionally active fragment thereof, or a polypeptide that is at least 80% homologous thereto.

2. A therapeutic composition according to claim 1 wherein components (a) and (b) are present in equal amounts.

3. A therapeutic composition according to claim 1 wherein components (a) and (b) are present in unequal amounts.

4. A therapeutic composition according to claims 2 or 3 wherein there is also provided a suitable excipient or carrier.

5. A therapeutic composition according to claims 2 or 3 wherein the composition is adapted for use in treating cancer.

6. A therapeutic composition according to claim 5 wherein the cancer is any one of the following: breast cancer, prostrate cancer, pancreatic cancer, kidney cancer, placental cancer, GI tract cancer, cancer of the testes or cancer of the ovaries.

7. A therapeutic composition comprising:
(a) An isolated, purified or recombinant nucleic acid molecule, encoding HAI-1, or a protein that is at least 80% homologous thereto, or that binds thereto under stringent hybridisation conditions; and
(b) An isolated, purified or recombinant nucleic acid molecule encoding HAI-2, or a protein that is at least 80% homologous thereto, or that binds thereto under stringent hybridisation conditions.

8. A therapeutic composition according to claim 7 wherein said nucleic acid molecule is any one of the following:
DNA, RNA, or a recombinant construct.

9. A therapeutic composition according to claim 8 wherein the nucleic acid molecule is incorporated in a vector.

10. A therapeutic composition according to claim 7 wherein the nucleic acid molecules comprise an associated promoter(s) that provides for either constitutive or inducible expression of said proteins.

11. A therapeutic composition according to claim 10 wherein HAI-1 and HAI-2 are under the control of the same promoter(s) and/or transcription factor(s) and are therefore either both constitutively expressed or inducibly expressed.

12. A therapeutic composition according to claim 10 wherein the expression of HAI-1 and HAI-2 is under the control of either a single promoter or the same promoter is operatively linked to each of the HAI-1 and HAI-2 coding sequences.

13. A therapeutic composition according to claim 10 wherein each nucleic acid molecule encoding HAI-1 or HAI-2 has its own, different promoter.

14. A therapeutic composition according to claim 7 wherein said nucleic acid molecules encoding HAI-1 and HAI-2 are adapted or designed for expression in a selected host system.

15. A therapeutic composition according to claim 14 wherein said host system is mammalian, bacterial or yeast.

16. A therapeutic composition according to claim 7 wherein the molecule encoding HAI-1 and/or HAI-2 is provided with a secretion signal whereby, following expression of the relevant protein, the protein is targeted for secretion.

17. A host cell transformed or transfected with the therapeutic composition according to claim 7.

18. A method for the production of a therapeutic composition according to claims 1 or 7 wherein:
(a) a host cell is transformed or transfected with a therapeutic composition according to claim 7;
(b) the host cell is cultured under conditions which enable expression of the said therapeutic composition; and
(c) the expression products HAI-1 and HAI-2 are then harvested.

19. A method according to claim 18 wherein said host cell is transformed or transfected with a therapeutic composition wherein the molecule encoding HAI-1 and/or HAI-2 is provided with a secretion signal whereby, following expression of the relevant protein, the protein is targeted for secretion and so part (c) of the methodology involves harvesting the expression products from the cell culture medium.

20. A method for preparing a therapeutic composition according to claim 1 comprising: (a) obtaining HAI-1 and/or HAI-2 protein from a suitable source of mammalian tissue and, where each protein is isolated from a separate source, mixing the two isolated products there together.

21. A method for the preparation of a therapeutic composition according claim 1 comprising:
(a) initiating, facilitating or enhancing the endogenous expression of HAI-1 and HAI-2 in either cancerous-tissue, pre-cancerous tissue or tissue adjacent or in the region of any of the aforementioned tissue.

22. A method according to claim 21 wherein said expression is initiated, facilitated or enhanced by activating the promoter of the HAI-1 and/or a HAI-2 gene.

23. A method according to claim 18 wherein further the nucleic acid molecule encoding HAI-1 and 1 or HAI-2 is amplified, using conventional techniques, in order to increase the production of HAI-1 or and/or HAI-2.

24. Primers for the use in the method according to claim 23.

25. A method according to claim 20 wherein further the nucleic acid molecule encoding HAI-1 and/or HAI-2 is amplified, using conventional techniques, in order to increase the production of HAI-1 or and/or HAI-2.

26. Primers for the use in the method according to claim 25.

27. A method according to claim 21 wherein further the nucleic acid molecule encoding HAI-1 and/or HAI-2 is amplified, using conventional techniques, in order to increase the production of HAI-1 or and/or HAI-2.

28. Primers for the use in the method according to claim 27.

29. A method of treating cancer comprising administering to an individual to be treated a therapeutic composition according to any one of claims 1 or 7.

30. An antibody raised against the HAI-2 component, or a combination of the HAI-1, HAI-2 components, of the therapeutic composition according to claim 1.
